(19) United States
(12) Patent Application Publication
(10) Pub. No.: US 2009/0306730 A1
(43) Pub. Date: Dec. 10, 2009

(54) DEFIBRILLATOR WITH HOUSING ARRANGEMENT AND SUPPORT DEVICE

(76) Inventor: Bruno Roso, Bad Dürrheim (DE)

Correspondence Address: PAULEY PETERSEN & ERICKSON, 2800 WEST HIGGINS ROAD, SUITE 365, HOFFMAN ESTATES, IL, 60169, US

(21) Appl. No.: 12/448,059

(22) PCT Filed: Dec. 7, 2007
(86) PCT No.: PCT/EP2007/010639

§ 371 Date: Jun. 5, 2009

(30) Foreign Application Priority Data
Dec. 7, 2006 (DE) 20 2006 018 673.5

(51) Int. Cl. A61N 1/39

(52) U.S. Cl. 607/5

(57) ABSTRACT

A defibrillator for permanent external application to a patient, with a protective shell arrangement receiving a base unit with defibrillator components, an electrode arrangement connected or connectable to the shell arrangement, and a support device. An ergonomic adaptation to the person wearing the defibrillator is achieved by the defibrillator components being spatially combined into subsidiary modules, and the shell arrangement has several subsidiary shells to which the subsidiary modules are distributed and which are movable relative to one another and connected to one another mechanically and electrically.

### BACKGROUND OF THE INVENTION

1. Field of the Invention

This invention relates to a defibrillator for permanent external attachment to a patient, having a covering arrangement that contains and protects a base unit with defibrillator components, having an electrode arrangement connected or connectable to this covering arrangement, and having a support device.

2. Discussion of Related Art

U.S. Pat. No. 4,576,170 discloses a defibrillator of this type that can be worn by a patient to be monitored. The overall system has two separate functional units, a monitoring part for monitoring cardiac activity and a defibrillator part, which are electrically connected to each other by connecting lines. An overall system with a defibrillator can cause discomfort in various situations in life such as when a person is moving or sleeping, particularly because the housing containing the defibrillator electronics is relatively bulky and uncomfortable to wear, even when having desired miniaturization.

The wearable defibrillators disclosed in German Patent Reference DE 697 17 192 T2 and U.S. Patent Application Publication 2003/0216787 A1 involve similar issues.

German Patent Reference DE 103 06 953 describes a support garment for positioning EKG measuring electrodes, EKG monitoring electrodes, and/or therapy electrodes as well as a cabling associated with them. A garment belt can have an elastic belt part that can be fastened in sliding fashion to an organizing plate, which can hold the measuring electrodes against the skin of the patient. German Patent Reference DE 691 21 470 discloses a belt-like electrode arrangement and German Patent Reference DE 689 27 898 discloses an electrode belt and a device attached to it.

The concepts for the design of the defibrillators themselves are continuously progressing toward the smallest possible monolithic embodiment of the defibrillator electronics to which only the electrodes or at most an additional energy source with batteries are attached via corresponding connecting cables. But because high energies (150 J to 250 J) and high voltages (up to 2000 V) are required to produce therapeutically effective shocks, the devices can only be miniaturized within limits. Even with a very high packing density, housing volumes of 300 to 500 cm³ are required.

### SUMMARY OF THE INVENTION

One object of this invention is to provide a defibrillator of the type mentioned at the beginning that offers the patient an improved wearing comfort, even over the long term.

This object is attained with a defibrillator having defining characteristics described in this specification and in the claims. The defibrillator components are spatially combined into submodules and the covering arrangement has a plurality of secondary covers over which the submodules are distributed and which are mechanically and electrically coupled to one another so that they are able to move in relation to one another.

This combination can, for example, be worn around the hips or around the chest. It can also be incorporated into a vest to be worn on the upper third of the body. Particularly when integrated into a vest, the combination can also be divided into a plurality of smaller combinations, for example each with three modules.

The division of the defibrillator components into individual submodules produces an ergonomically convenient and aesthetic wearing of this device on the body in all situations in life, when awake, sleeping, or moving.

In some embodiments the secondary covers are arranged in a plane or in a row.

In other embodiments, for accommodating the submodules of the defibrillator electronics in a protective way, the secondary covers are embodied in the form of rigid housing modules or at least partially in the form of flexible protective coverings.

Various advantageous steps can be taken to adapt to the shape of the body so that the secondary covers are mechanically coupled to one another by flexible intermediate pieces or by articulated pieces that engage with one another.

A design that is advantageous for wearing comfort is also achieved if the secondary covers are attached to a flexible support material.

If the flexible support material is entirely or partially composed of a textile, then it is possible to fulfill various requirements such as gentleness to skin at a variety of outside temperatures or with a variety of movement requirements.

The wearing comfort is improved because insulated electrical connecting lines are integrated into the intermediate pieces or into the support material.

In other various advantageous embodiment options, the electrical connecting lines are embodied in the form of textile electrical lines or flexible printed circuit boards.

The measures according to which the electrical connecting lines are at least partially embodied in the form of a bus system contribute to an advantageous electrical data transmission and low wiring complexity.

Other advantages of dividing the base unit into a combination of submodules include the following. The individual modules are small, flat, and movable in relation to one another, and thus it is possible to achieve a high degree of wearing comfort. Because the submodules are flat, they can be worn almost invisibly under the clothes. Changing the number or height of the modules makes it possible to adapt the volume to the actual requirements. The connecting technique using flat conductors, for example, assures favorable electrical properties, low impedance, low inductance, simple insulation by means of intermediate layers. Appropriate dimensioning and the use of suitable plastics make it possible to achieve a high degree of ruggedness of the system and the connecting points. The submodules can be connected to one another in a watertight fashion by producing the composite in one piece or by welding them to one another.

As known, a defibrillator is used for therapy of life-threatening cardiac arrhythmia and in particular comprises an energy storage unit composed of one or more capacitors, a charging device for this energy storage unit, a switch arrangement for forming the defibrillation pulse, and a set of control electronics. These defibrillator components can be embodied in the form of the individual submodules. It is also possible to subdivide the defibrillator components further or to combine them, thus yielding a number of submodules that differs from the number of components.

An exemplary embodiment of an externally wearable defibrillator involves the following circumstances, for example. Emitting effective defibrillator pulses requires a pulse energy of approximately 150 J. Assuming that these pulses have traditional curve shapes, this requires approximately 250 J to be stored ahead of time in a capacitor used as an energy storage unit. Such a capacitor requires a volume of approximately 150 cm³. If this capacitor is divided into 4 equal parts, then this results in a volume of 38 cm³ per part. A square base surface of 7*7 cm² would intrinsically require a height of approximately 7.5 mm for the capacitor. Including the cover and connecting elements, a capacitor module of this kind could thus be produced in a volume of approximately 8*9*1.2 cm³. A battery pack required to supply energy to the entire system can likewise be accommodated in one or if need be two such modules. The electronics required for producing a pulse in a biphasic truncated exponential (BTE) form on this base surface is also possible. A complete external defibrillator can thus be composed of 6 to 7 of the above-mentioned submodules. If these submodules are connected to form a composite unit, for example a chain, then this can be worn, for example, around the hips or around the ribcage without causing excessive discomfort to the wearer. The wearing comfort and therefore acceptance can be further increased through additional measures such as cushioning or a concave shape.

In constructing a defibrillator according to this invention, it is advantageous to use flat capacitors, flat battery packs, flat cables or flexible printed circuit boards, and the flattest possible electronics components, such as power semiconductors that have no housing and are mounted on a ceramic substrate, to mount the above-mentioned components in flat plastic cups, to connect these plastic cups to one another by flexible plastic bands that are attached to two opposing sides, to embody the electrical connection between the components contained in the cups by flat conductors that are encased in the flexible plastic bands and/or to electrically and mechanically connect all of the submodules of the defibrillator electronics into a combination of secondary covers arranged in a plane or a row.

### BRIEF DESCRIPTION OF THE DRAWINGS

This invention is explained in detail below in view of exemplary embodiments with reference to the drawings, wherein:

FIG. 1 shows a diagrammatic view of a first embodiment for submodules of a set of defibrillator electronics arranged in a flexible combination, with respective secondary covers in a planar arrangement;

FIG. 2 shows a diagrammatic view of another embodiment for a flexible combination of submodules of a set of defibrillator electronics situated in respective secondary covers arranged in a row;

FIG. 3 shows a diagrammatic view of another embodiment for a series arrangement of submodules of a set of defibrillator electronics in respective secondary covers in a flexible combination arranged in chain fashion; and

FIGS. 3A and 3B each shows a more detailed depiction of a flexible combination of submodules of a defibrillator arrangement in respective secondary covers arranged in a chain fashion.

### DETAILED DESCRIPTION OF THE INVENTION

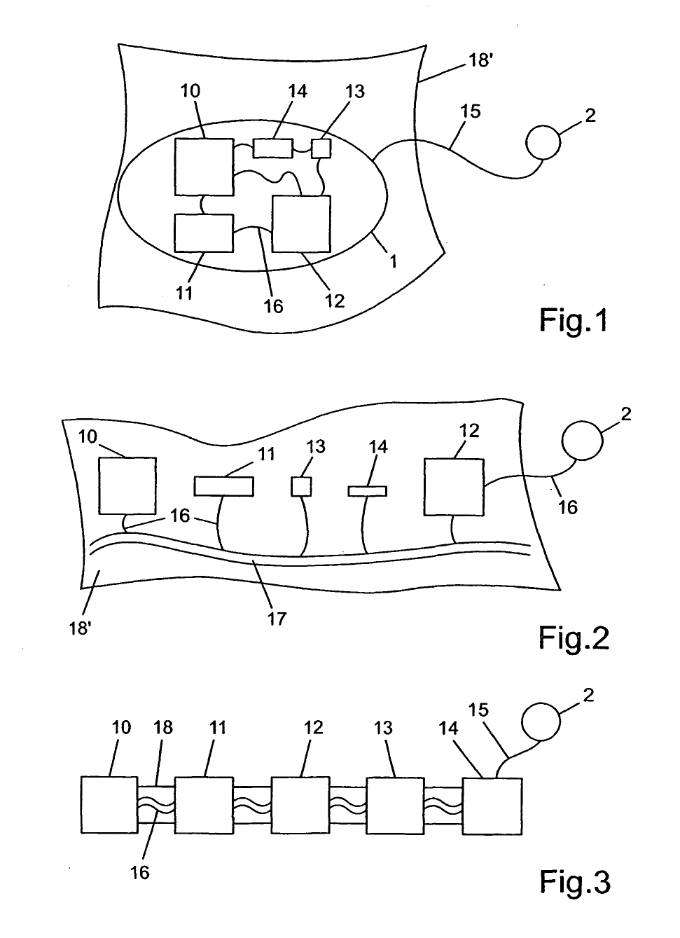

FIG. 1 schematically shows a defibrillator, having a base unit 1 that contains a set of defibrillator electronics, with an electrode arrangement 2 connected to it via a connecting line 15, which arrangement includes defibrillation electrodes and can also have measurement electrodes if needed or an integrated sensor system for measurement signals to be picked up from the patient, for example EKG signals, as is intrinsically customary in such defibrillators that can be externally worn by a patient. The connecting line 15, which has a number of wires, is electrically connected to the base unit 1 either permanently or by a plug.

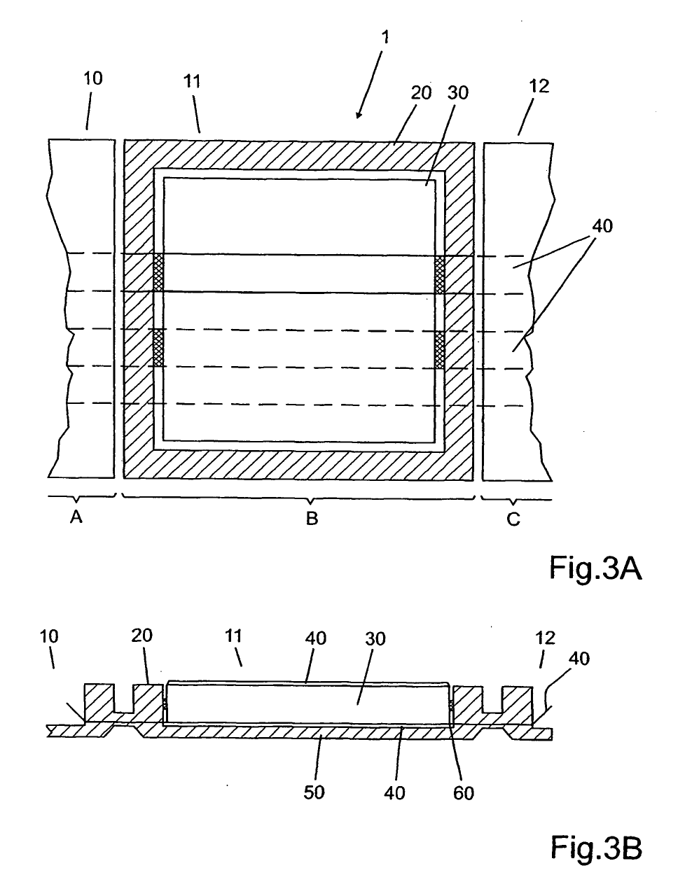

The set of defibrillator electronics of the base unit 1 is divided into a plurality of submodules that are advantageously associated with various defibrillation components such as an energy storage unit with one or more capacitors, a charging device for the energy storage unit, a switch arrangement for forming the defibrillation pulse, a set of control electronics, an EKG amplifier, a signal analysis circuit, an HV output stage, and a user interface. Additional components can include, for example, communications modules (GSM modules, UMTS modules), a GPS module for locating the patient, or components of a human/machine interface. The submodules are connected to one another electrically via connecting lines 16 and mechanically via a flexible coupling such as a flexible support 18′, accommodated in respective secondary covers 10, 11, 12, 13, 14, and form a combination that is interconnected in a functionally reliable fashion via the flexible electrical and mechanical coupling and that is arranged in a planar form in the exemplary embodiment shown in FIG. 1. FIG. 2 shows an exemplary embodiment for a combination of submodules, with the secondary covers 10, 11, 12, 13, 14 arranged in a row in which the submodules are connected via connecting lines 16 to a bus line 17 with a suitable number of wires and are mechanically coupled, for example, by a flexible support 18′. In the additional exemplary embodiment according to FIG. 3, the submodules with their secondary covers 10, 11, 12, 13, 14 are mechanically connected to one another in a chain-link fashion by the mechanical coupling 18, which are embodied in the form of flexible coupling means such as flexible plastic, a textile, or elements that are hooked to one another and are electrically connected to one another via connecting lines 16.

The individual secondary covers 10, 11, 12, 13, 14 can be embodied in various ways, for example as strong, relatively rigid flat plastic housings or as flexible receiving pockets made of a plastic material and/or woven material. They can be suitably adapted in shape and with regard to body compatibility and are designed as flexible and/or soft in order to adapt to various situations in life, such as for good mobility or for a person who is resting. In addition, the material can be suitably selected for gentleness to skin, for absorption of body temperature or perspiration, or for allowing said temperature or perspiration to pass through. In addition, a moisture protection for the electronics contained in the secondary covers can be achieved by correspondingly moisture-proof material and moisture-proof closures. On the whole, it is thus possible on the one hand to assure a high degree of wearing comfort and on the other, to assure a reliable functionality.

FIG. 3 shows a series-arrangement of the secondary covers 10, 11, 12, 13, 14, which are electrically connected to one another via electrical connecting lines 16 and are mechanically coupled to one another in chain-link fashion via elastic or interlinked coupling means 18.

Advantageous embodiments can be achieved through special embodiments of the installed components, such as by using flat capacitors without housings, by varying the chain, such as by subdividing the link chain, by installing the components in a vest, or by forming them into a two-dimensional chain link lattice.

FIGS. 3A and 3B schematically show more details of a series arrangement in the form of a link chain, with essential components of the structure, including an element housing 1 with a plurality of secondary covers in the form of housing modules A, B, C (chain link B such as for a capacitor block 30) that are movably coupled to one another in the form of a chain. For example, on a supporting base 50, the chain link B has a cup-shaped bottom part 20 for a capacitor block 30 and the electrical connection is produced by flat conductors 40 with appropriate contact points 60.

By way of example, FIGS. 3A and 3B show the embodiment of a still-open housing module B with an appropriate submodule for a capacitor block and its connection to the two adjacent modules A and C. The drawings do not show the still-needed closure of the submodule by a for example welded cover. Correspondingly, the housing module can also accommodate other components selected from among those mentioned above or from still other components.

In an arrangement of the submodules of the defibrillator electronics in housing modules A, B, C that are connected to form such a link chain, a belt that is formed with the link chain can also be used, for example, to position electrodes.

The above-described embodiments of the defibrillator base unit 1 with the submodules, which are contained in the secondary covers 10, 11, 12, 13, 14 and are connected to one another to form a flexible mechanical combination via the flexible mechanical coupling means and electrical connections, achieve a spatial separation of individual functional units of the main defibrillator devices so that the defibrillator is convenient to wear, particularly over the long term. It is also possible for individual functional units, such as the main voltage capacitor arrangement, to be subdivided to a greater or lesser degree into a plurality of modules. On the whole, this achieves an ergonomic adaptation of the defibrillator as a composite system to the body of the wearer.

An embodiment in which the submodules are mounted onto a flexible support material 18′, such as made of a cohesive flexible plastic or fiber composite such as a textile, likewise permits a planar arrangement or series arrangement of the submodules, thus permitting an ergonomically favorable adaptation to the body of the wearer. The electrical connecting lines of the submodules can be integrated into the support material 18′, for example by textile cables with stable electrical properties or by flexible printed circuit boards. In an advantageous embodiment, the support material 18′ can be in the form of a protective cover and the secondary covers can be in the form of pockets in the support material so that the electrical connecting means and/or the defibrillator electronics are accommodated in them in a protective fashion. Here, too, it is possible to produce the electrical connection to a bus system with definite lines that are separate from the high-voltage lines and can then be contacted individually by the submodules when they are attached support material. The flexible arrangement of the individual submodules in the composite permits an advantageous ergonomic adaptation of the defibrillator system to the wearer.

1. A defibrillator for permanent external attachment to a patient, comprising a protective covering arrangement that contains and protects a base unit with defibrillator components, having an electrode arrangement connected or connectable to the covering arrangement, and having a support device, wherein the defibrillator components are spatially combined into submodules and the covering arrangement has a plurality of secondary covers (10, 11, 12, 13, 14) over which the submodules are distributed and which are mechanically and electrically coupled to one another and movable in relation to one another.

2. The defibrillator as recited in claim 1, wherein the secondary covers (10, 11, 12, 13, 14) are arranged in a plane or in a row.

3. The defibrillator as recited in claim 2, wherein the secondary covers (10, 11, 12, 13, 14) are rigid housing modules (A, B, C) or are at least partially embodied in a form of flexible protective coverings.

4. The defibrillator as recited in claim 3, wherein the secondary covers (10, 11, 12, 13, 14) are mechanically coupled to one another by flexible intermediate pieces or by articulated pieces that engage with one another.

5. The defibrillator as recited in claim 4, wherein the secondary covers (10, 11, 12, 13, 14) are attached to a flexible support material (18′).

6. The defibrillator as recited in claim 5, wherein the flexible support material (18′) is entirely or partially of a textile or a plastic film.

7. The defibrillator as recited in claim 5, wherein insulated electrical connecting lines (16) are integrated into the intermediate pieces or into the support material (18′).

8. The defibrillator as recited in claim 7, wherein the electrical connecting lines (16) are in a form of textile electrical lines or flexible printed circuit boards.

9. The defibrillator as recited in claim 8, wherein the electrical connecting lines (16) are at least partially embodied in a form of a bus system (17).

10. The defibrillator as recited in claim 1, wherein the secondary covers (10, 11, 12, 13, 14) are rigid housing modules (A, B, C) or are at least partially embodied in a form of flexible protective coverings.

11. The defibrillator as recited in claim 1, wherein the secondary covers (10, 11, 12, 13, 14) are mechanically coupled to one another by flexible intermediate pieces or by articulated pieces that engage with one another.

12. The defibrillator as recited in claim 1, wherein the secondary covers (10, 11, 12, 13, 14) are attached to a flexible support material (18′).

13. The defibrillator as recited in claim 12, wherein the flexible support material (18′) is entirely or partially of a textile or a plastic film.

14. The defibrillator as recited in claim 4, wherein insulated electrical connecting lines (16) are integrated into the intermediate pieces or into the support material (18′).

15. The defibrillator as recited in claim 14, wherein the electrical connecting lines (16) are in a form of textile electrical lines or flexible printed circuit boards.

16. The defibrillator as recited in claim 14, wherein the electrical connecting lines (16) are at least partially embodied in a form of a bus system (17).
